(19) United States
(12) Patent Application Publication
(10) Pub. No.: US 2005/0203175 A1
(43) Pub. Date: Sep. 15, 2005

(54) Parenteral composition of paracetamol

(76) Inventor: Ioulia Tseti, Kifissia (GR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/498,878

(22) PCT Filed: Dec. 18, 2001
(86) PCT No.: PCT/GR01/00047

§ 371 Date: Jun. 16, 2004

(51) Int. Cl. A61K 31/16

(52) U.S. Cl. 514/474; 514/629

(57) ABSTRACT

The present invention refers to a pharmaceutical preparation, comprising a novel, stable solution of Paracetamol for parenteral administration, useful to establish an analgesic and antipyretic effect.

The present invention refers to pharmaceutical composition comprising Paracetamol for parenteral administration.

Paracetamol is considered to be the main active metabolite of phenacetin and acetanidile having analgesic and antipyretic properties. Paracetamol has equivalent analgesic and antipyretic action to that of aspirin whilst it expresses weak anti-inflammatory action therefore its use in inflammatory rheumatic diseases is limited.

The mechanism of its analgesic action is still unclarified. It is believed that it mainly acts by inhibiting prostaglandins biosynthesis and to a lesser extent by peripherically inhibiting algogenic stimulus origin. The peripheral action is due also to inhibition of proglandins biosynthesis or to inhibition or to other endogenous substances action that sensitize pain's receptors after mechanic or chemical stimulation.

As far as its antipyretic action is concerned, Paracetamol induces temperature fall to feverish but not to normal subjects.

It is believed that the antipyretic effect of Paracetamol is due to central action on the temperature controlled centre of hypothalamus resulting in peripheral vasodilation leading to skin peripheral blood flow increase, perspiration and temperature loss.

This peripheral action of Paracetamol is due also to prostaglandins bio-synthesis inhibition into hypothalamus. Paracetamol administered in recommended dosage does not exert any effect of the cardiovascular and respiratory system nor provokes acid-base balance disorders.

Several studies have confirmed the effectiveness and safety of Paracetamol's parenteral administration.

Paracetamol is well absorbed when intramuscularly administered and its blood level is similar to that obtained after its oral administration.

The absorption rate is slower of that obtained when Paracetamol is orally administered, resulting in desirable blood levels for more prolonged time.

There is also another advantage of injectable Paracetamol since the 20% loss of the drug that is observed after oral administration doesn't exist (Macheras et al. 1989, Pharmaceutical Codex 1994).

Paracetamol is metabolized by the microsomal enzymes of the liver and 95% of it is excreted through urines as conjugated derivatives of sulfuric (35%) and glucouronic acids (60%) whilst only 2% is excreted unchangeable (Gillette 1981, Clissold 1986, Remington 1990, Insel 1992, AMA-DE 1994).

Also a small part of Paracetamol, approx. 3%, is oxidized by the liver cytochrome P-450 to a toxic intermediate metabolite that is connected to the liver deposit of glutathione, producing finally a non-toxic combination, which is excreted conjugated with cysteine and mercapturic acid (Mitchell et al. 1982, Jackson et at. 1984, Remington 1990, Insel 1992).

Therefore, Paracetamol parenteral solutions are indispensable for use in modern therapeutics for a greater and quicker therapeutic effect.

Whilst Paracetamol is soluble in many organic solvents, however solutions of Paracetamol with such solvents are unfit for therapeutical use, because of the produced toxicity when parenterally administered (intramuscularly or intravenously) and because of the present technical problems as i.e. chemical instability leading to precipitates, low fluidity etc.

As above, the preparations of injectable solutions of Paracetamol and combinations of Paracetamol with other active substances require the choice of the suitable solvent or combination of solvents, comprising also water, reciprocating to certain requirements of suitability as: to be pharmacologically inactive, to not form complexes with the active substance, to be blood conventional, free of sensitization or irritating activity, chemically stable, clear and not influenced by pH declinations.

Additionally, it is important the selected solvents to not interfere with Paracetamols' or other's substances therapeutical properties. From the pharmacotechnical point of view, the selected solvent or solvents system must have the full ability of mixing with water not only because this way it or they will facilitate the manufacturing process but will also reduce the manufacturing cost.

Furthermore, the absorption by the organism of the solution and the compatibility with the human blood are of high importance. Moreover, chemical stability is highly related to the antioxidant properties of the injectable solution.

Documents GR-B-871 510, GR-B-1 001 523, GR-B-1 002 731 and EP application number 97 600 009 are related to injectable parenteral solutions including Paracetamol dissolved in Ethanol, Glycerol formal and Water. However, none of the said prior art documents combines both the presence of Nipagin A and Nipasol M as antioxidant together with Paracetamol as the only pharmaceutically active as in the present invention, as defined by the appended claims, in particular independent claim 1.

### DETAILED DESCRIPTION OF THE INVENTION

The present invention is defined by the appended claims.

The claimed solutions overcome all the above problems of the prior art, i.e. they are chemically stable, clear, non-toxic, do not participate, show high fluidity, do not form complexes, are blood conventional, free of sensitization or irritating activity, are not influenced by pH declinations, are well absorbed by the organism of human beings, are very well compatible with the human blood, resist oxidation better than all previous similar solutions in particular those comprising further pharmaceutical actives, are easy to produce, the organic solvents are fully mixing with water, show improved pharmacokinetic properties, show improved bio-availability and local tolerance in the site of injection.

Since Paracetamol is practically insoluble in water, efforts made for its dissolution into organic-solvents or mixtures of them, suitable for parenteral use.

Paracetamol is soluble in Methanol, Ethanol, DMF, Ethylene chlorine, Benzyl ethanol and other organic solvents, but none of them can be used alone or in a mixture, because of their toxicity. Our experiments showed finally that the qualified solvent in the case of Paracetamol was Glycerol formal.

Glycerol formal is an almost atoxic solvent (LD50 I.V. to rats, 3.5 mg/kg body weight) possesses the advantage of mixing with Water, Alcohol and Propylene Glycol and has been proved to be the most favourable and qualified solvent for Paracetamol's injectable parenteral solutions, which can be used alone or in mixtures with water, Ethanol, Benzyl ethanol and Propylene glycol.

Further Compounds

One or more further additive compounds can also be included in the invented composition.

Such additives can be Lidocaine HCl, pharmaceutical active showing the advantage to attenuate pain at the site of injection, Disodium Phosphate, Sodium hydroxide, Sodium carbonate or Disodium Citrate to adjust pH to 5-6.5, preferably to 5.5-6 even more preferably to 5.5, Disodium Edetate as chelating agent, Nipagin A and Nipasol M as antioxidant and other adjusting to the constituents antioxidant agents.

### EXAMPLES

The following examples are according to the present invention as defined by independent claim 1 and show all the above nentioned advantages.

The advantages include improved antioxidant properties and absorption properties when compared either with the same solution but without the antioxidant mixture Nipagin A and Nipasol M or when said antioxidant mixture is fully or partially replaced by an other antioxidant such as Sodium metabisulfite, derivatives of Ascorbic acid, derivatives carriers of Thiol group and/or Butyl Hydroxy Anisol or when compared with the same solution including further pharmaceutical actives additionally to Paracetamol such as the spasmolytic Hyoscine-N-Butylbromide the central antalgic Codeine Phosphate or any synthetic or semi-synthetic morphinic analgesic, the myorelaxants Carisoprodol and Orphenadrine citrate, the anti-oxidant Acetyl-cysteine, the analgesic Acetylsalicylic acid, Caffeine and pharmaceutically accepted combinations of them with Paracetamol.

### Example 1

| Constituents | Quantities | |
|---|---|---|
| Paracetamol | 150.00 | mg |
| Lidocaine HCl | 5.00 | mg |
| Disodium Edetate | 0.50 | mg |
| Disodium Phosphate | q.s. to pH 5-5.5 | |
| Nipagin A | 1.80 | mg |
| Nipasol M | 0.20 | mg |
| Glycerol formal | 0.75 | ml |
| Ethanol | 0.15 | ml |

| | |
|---|---|
| Water for injection | q.s. to 1.00 ml |

### Example 2

| Constituents | Quantities | |
|---|---|---|
| Paracetamol | 150.00 | mg |
| Lidocaine HCl | 5.00 | mg |
| Disodium Edetate | 0.50 | mg |
| Disodium Phosphate | q.s. to pH 5-5.5 | |
| Butyl Hydroxy Anisol | 0.20 | mg |
| Ascorbic acid | 0.50 | mg |
| Glycerol formal | 0.75 | ml |
| Ethanol | 0.15 | ml |

| | |
|---|---|
| Water for injection | q.s. to 1.00 ml |

### Example 3

| Constituents | Quantities | |
|---|---|---|
| Paracetamol | 150.00 | mg |
| Lidocaine HCl | 5.00 | mg |
| Disodium Edetate | 0.50 | mg |
| Disodium Phosphate | q.s. to pH 5-5.5 | |
| Sodium metabilulfite | 1.00 | mg |
| Glycerol formal | 0.75 | ml |
| Ethanol | 0.15 | ml |

| | |
|---|---|
| Water for injection | q.s. to 1.00 ml |

### Example 4

| Constituents | Quantities | |
|---|---|---|
| Paracetamol | 150.00 | mg |
| Lidocaine HCl | 5.00 | mg |
| Disodium Edetate | 0.50 | mg |
| Disodium Phosphate | q.s. to pH 5-5.5 | |
| Nipagin A | 1.80 | mg |
| Nipasol M | 0.20 | mg |
| Glycerol formal | 0.70 | ml |
| Propylene glycol | 0.20 | ml |

| | |
|---|---|
| Water for injection | q.s. to 1.00 ml |

### Example 5

| Constituents | Quantities | |
|---|---|---|
| Paracetamol | 150.00 | mg |
| Hyoscine-N-Butylbromide | 5.00 | mg |
| Lidocaine HCl | 5.00 | mg |
| Disodium Edetate | 0.50 | mg |
| Sodium hydroxide | q.s. to pH 5-5.5 | |
| Nipagin A | 1.80 | mg |
| Nipasol M | 0.20 | mg |
| Glycerol formal | 0.75 | ml |
| Ethanol | 0.15 | ml |

| | |
|---|---|
| Water for injection | q.s. to 1.00 ml |

### Example 6

| Constituents | Quantities | |
|---|---|---|
| Paracetamol | 120.00 | mg |
| Codeine Phosphate as sulphate | 6.50 | mg |
| Lidocaine HCl | 5.00 | mg |
| Disodium Edetate | 0.50 | mg |
| Disodium Phosphate | q.s. to pH 5-5.5 | |
| Butyl Hydroxide Anisol | 0.20 | mg |
| Ascorbic acid | 0.50 | mg |
| Glycerol formal | 0.75 | ml |
| Propylene glycol | 0.20 | ml |

| | |
|---|---|
| Water for injection | q.s. to 1.00 ml |

### Example 7

| Constituents | Quantities | |
|---|---|---|
| Paracetamol | 100.00 | mg |
| Carisoprodol | 50.00 | mg |
| Lidocaine HCl | 5.00 | mg |
| Disodium Edetate | 0.50 | mg |
| Sodium hydroxide | q.s. to pH 5-5.5 | |
| Nipagin A | 1.80 | mg |
| Nipasol M | 0.20 | mg |
| Glycerol formal | 0.75 | ml |
| Ethanol | 0.15 | ml |

| | |
|---|---|
| Water for injection | q.s. to 1.00 ml |

### Example 8

| Constituents | Quantities | |
|---|---|---|
| Paracetamol | 60.00 | mg |
| Acetylsalicylic acid | 100.00 | mg |
| Caffeine | 10.00 | mg |
| Lidocaine HCl | 5.00 | mg |
| Sodium hydroxide | q.s. to pH 5-5.5 | |
| Disodium Edetate | 0.50 | mg |
| Nipagin A | 1.80 | mg |
| Nipasol M | 0.20 | mg |
| Ascorbic acid | 0.50 | mg |
| Glycerol formal | 0.80 | ml |
| Propylene glycol | 0.10 | ml |

| | |
|---|---|
| Water for injection | q.s. to 1.00 ml |

1. Pharmaceutical injectable parenteral solution comprising a) Paracetamol as the only pharmaceutically active, b) a mixture of solvents comprising Ethanol, Glycerol formal and Water and c) the antioxidant mixture Nipagin A (is the Trade Name of CLARIANT UK LTD company and corresponds to Methyl-parahydroxy benzoate) and Nipasol M (is the Trade Name of CLARIANT UK LTD company and corresponds to Propyl-parahydroxy benzoate).

2. Solution according to claim 1 wherein the ratio ethanol:glycerol formal:water is 5-15:60-80:5-10 by volume.

3. Solution according to claim 1 or 2 containing one or more antioxidant agents such as Sodium metabisulphite, derivatives of Ascorbic acid, derivatives carriers of Thiol group or Butyl Hydroxide Anisol.

4. Solution according to claim 1 or 2 containing one or more of Sodium hydroxide, Sodium carbonate, Trisodium citrate, Disodium phosphate to achieve a pH of 5-6.5.

5. Solution according to claim 1 or 2 containing Disodium edetate.

6. Solution according to claim 1 or 2 containing Benzyl alcohol or Propylene glycol.

7. Solution according to claim 1 or 2 containing one or more pharmaceutical actives such as Hyoscine-N-Butylbromide, Codeine phosphate or Sulfate, Carisoprodol, Orphenadrine citrate, Acetylsalicylic acid, Caffeine, synthetic or hemi-synthetic morphinic derivatives.

8. Solution according to claim 1 comprising 150 mg Paracetamol, 5 mg Lidocaine HCl, 0.40 mg Disodium phosphate, 0.50 mg Disodium Edetate, 1.8 mg Nipagin A, 0.20 mg Nipasol M, 0.75 ml Glycerol formal, 0.15 ml Ethanol and Water for injection q,s. ad 1 ml.

9. Solution according to claim 1 wherein the ratio Glycerol formal-Propylene glycol-Water is 60-80:20-40:5-15 by volume.

10. Solution according to claim 1 wherein the ratio Glycerol formal-Benzyl alcohol-Water is 80:10:10 by volume.

11. Pharmaceutical injectable parenteral solution comprising a) Paracetamol as the only pharmaceutically active, b) a mixture of solvents comprising Ethanol, Glycerol formal and Water and c) the antioxidant mixture Nipagin A (is the Trade Name of CLARIANT UK LTD company and corresponds to Methyl-parahydroxy benzoate) and Nipasol M (is the Trade Name of CLARIANT UK LTD company and corresponds to Propyl-parahydroxy benzoate), wherein the ratio ethanol: glycerol formal:water is 5-15:60-80:5-10 by volume.

12. Solution according to claim 11 containing one or more antioxidant agents such as Sodium metabisulphite, derivatives of Ascorbic acid, derivatives carriers of Thiol group or Butyl Hydroxide Anisol.

13. Solution according to claim 11 or 12 comprising containing one or more of Sodium hydroxide, Sodium carbonate, Trisodium citrate, Disodium phosphate to achieve a pH of 5-6.5.

14. Solution according to claim 11 or 12 containing Disodium edetate.

15. Solution according to claim 11 or 12 comprising containing Benzyl alcohol or Propylene glycol.

16. Solution according to claim 11 or 12 containing one or more pharmaceutical actives such as Hyoscine-N-Butylbromide, Codeine phosphate or Sulfate, Carisoprodol, Orphenadrine citrate, Acetylsalicylic acid, Caffeine, synthetic or hemi-synthetic morphinic derivatives.

17. Pharmaceutical injectable parenteral solution comprising 150 mg Paracetamol, 5 mg Lidocaine HCl, 0.40 mg Disodium phosphate, 0.50 mg Disodium Edetate, 1.8 mg Nipagin A (is the Trade Name of CLARIANT UK LTD company and corresponds to Methyl-parahydroxy benzoate), 0.20 mg Nipasol (is the Trade Name of CLARIANT UK LTD company and corresponds to Propyl-parahydroxy benzoate), 0.75 ml Glycerol formal, 0.15 ml Ethanol and Water for injection q.s. ad 1 ml.

18. Pharmaceutical injectable parenteral solution comprising a) Paracetamol as the only pharmaceutically active, b) a mixture of solvents comprising Ethanol, Glycerol formal and Water and c) the antioxidant mixture Nipagin A (is the Trade Name of CLARIANT UK LTD company and corresponds to Methyl-parahydroxy benzoate) and Nipasol M (is the Trade Name of CLARIANT UK LTD company and corresponds to Propyl-parahydroxy benzoate), wherein the ratio Glycerol formal-Propylene glycol-Water is 60-80:20-40:5-15 by volume.

19. Pharmaceutical injectable parenteral solution comprising a) Paracetamol as the only pharmaceutically active, b) a mixture of solvents comprising Ethanol, Glycerol formal and Water and c) the antioxidant mixture Nipagin A (is the Trade Name of CLARIANT UK LTD company and corresponds to Methyl-parahydroxy benzoate) and Nipasol M (is the Trade Name of CLARIANT UK LTD company and corresponds to Propyl-parahydroxy benzoate), wherein the ratio Glycerol formal-Benzyl alcohol-Water is 80:10:10 by volume.

20. Pharmaceutical injectable parenteral solution comprising a) Paracetamol as the only pharmaceutically active or in combination with further compounds comprising one or more pharmaceutical actives such as Hyoscine-N-Butylbromide, Codeine phosphate or Sulfate, Carisoprodol, Orphenadrine citrate, Acetylsalicylic acid, Caffeine, synthetic or hemi-synthetic morphinic derivatives, b) a mixture of solvents comprising Ethanol, Glycerol formal and Water and c) the antioxidant mixture Nipagin A and Nipasol M wherein the ratio Glycerol formal-Propylene glycol-Water is 60-80:20-40:5-15 by volume.

21. Pharmaceutical injectable parenteral solution comprising a) Paracetamol as the only pharmaceutically activeor in combination with further compounds comprising one or more pharmaceutical actives such as Hyoscine-N-Butylbromide, Codeine phosphate or Sulfate, Carisoprodol, Orphenadrine citrate, Acetylsalicylic acid, Caffeine, synthetic or hemi-synthetic morphinic derivatives, b) a mixture of solvents comprising Ethanol, Glycerol formal and Water and c) the antioxidant mixture Nipagin A (is the Trade Name of CLARIANT UK LTD company and corresponds to Methyl-parahydroxy benzoate) and Nipasol M (is the Trade Name of CLARIANT UK LTD company and corresponds to Propyl-parahydroxy benzoate), wherein the ratio Glycerol formal-Benzyl alcohol-Water is 80:10:10 by volume.

22. Solution according to claim 2, wherein the ratio is 15:75:10 by volume.

23. Solution according to claim 4, wherein the pH is 5.5-5.6.

24. Solution according to claim 9, where the ratio is 70:20:10 by volume.

25. Solution according to claim 13, wherein the pH is 5.5-5.6.
